(19) United States
(12) Patent Application Publication
(10) Pub. No.: US 2011/0300605 A1
(43) Pub. Date: Dec. 8, 2011

(54) NANOSCALING ORDERING OF HYBRID MATERIALS USING GENETICALLY ENGINEERED MESOSCALE VIRUS

(75) Inventors: ANGELA M. BELCHER, LEXINGTON, MA (US); SEUNG-WUK LEE, AUSTIN, TX (US)

(73) Assignee: BOARD OF REGENTS, THE UNIVERSITY OF TEXAS SYSTEM

(21) Appl. No.: 13/193,884

(22) Filed: Jul. 29, 2011

Related U.S. Application Data
(62) Division of application No. 11/987,673, filed on Dec. 3, 2007, now abandoned.
Application No. 11/987,673 is a continuation of application No. 10/157,775, filed on May 29, 2002, now abandoned.
(60) Provisional application No. 60/326,583, filed on Oct. 2, 2001.

(51) Int. Cl. C12N 7/00; B82Y 99/00

(52) U.S. Cl. 435/235.1; 977/773; 977/811

(57) ABSTRACT

The present invention includes methods for producing nanocrystals of semiconductor material that have specific crystallographic features such as phase and alignment by using a self-assembling biological molecule that has been modified to possess an amino acid oligomer that is capable of specific binding to semi-conductor material. One form of the present invention is a method to construct ordered nanoparticles within the liquid crystal of the self-assembling biological molecule.

### CROSS-REFERENCE TO RELATED APPLICATIONS

This application is a divisional of U.S. application Ser. No. 11/987,673, filed Dec. 3, 2007, which is a continuation of U.S. application Ser. No. 10/157,775, filed May 29, 2002, which claims priority to U.S. Provisional Application No. 60/326,583, filed Oct. 2, 2001. The entire contents of each of the aforementioned applications are incorporated herein by reference.

The research carried out in the subject application was supported in part by a grant from the National Science Foundation (Grant No. NSF NIRT CTS-0103473). The U.S. Government has rights in this invention.

### TECHNICAL FIELD OF THE INVENTION

The present invention is directed to organic materials capable of binding to inorganic materials and specifically, toward bacteriophage that can bind semiconductor materials and form well-ordered structures.

### BACKGROUND OF THE INVENTION

In biological systems, organic molecules exert a remarkable level of control over the nucleation and mineral phase of inorganic materials such as calcium carbonate and silica, and over the assembly of building blocks into complex structures required for biological function.

Materials produced by biological processes are typically soft, and consist of a surprisingly simple collection of molecular building blocks (i.e., lipids, peptides, and nucleic acids) arranged in astoundingly complex architectures. Unlike the semiconductor industry, which relies on a serial lithographic processing approach for constructing the smallest features on an integrated circuit, living organisms execute their architectural “blueprints” using mostly non-covalent forces acting simultaneously upon many molecular components. Furthermore, these structures can often elegantly rearrange between two or more usable forms without changing any of the molecular constituents.

The use of “biological” materials to process the next generation of microelectronic devices provides a possible solution to resolving the limitations of traditional processing methods. The critical factors in this approach are identifying the appropriate compatibilities and combinations of biological-inorganic materials, and the synthesis of the appropriate building blocks.

### SUMMARY OF THE INVENTION

The present inventors have designed constructs and produced biological materials that direct and control the assembly of inorganic materials into controlled and sophisticated structures. The use of biological materials to create and design materials that have interesting electrical or optical properties may be used to decrease the size of features and improve the control of, e.g., the opto-electical properties of the material. Semiconductor materials are typically made from zinc sulfide, gallium arsenide, indium phosphate, cadmium sulfide, aluminum arsenide aluminum stibinide and silicon. These semiconductor materials are often classified into Group III-Group V and Group II-Group VI semiconductor materials.

Organic-inorganic hybrid materials offer new routes to novel materials and devices. The present inventors have exploited the organic-inorganic hybrids to select for peptides that can bind to semiconductor materials. Size controlled nanostructures give optically and electrically tunable properties of semiconductor materials. Using the present invention, organic additives have been used to modify the inorganic morphology, phase, and nucleation direction of semiconductor materials. The monodispersed nature of biological materials makes the system compatible for highly ordered smectic-ordering structure.

Building well-ordered and well-controlled two- and three-dimensional structures at the nanolength scale is the major goal of building next generation optical, electronic and magnetic materials and devices. Many researchers have focused on building such structures using traditional materials approaches. As disclosed herein, the present inventors have demonstrated that soft materials can act as self-organizers that organize inorganic materials at the nanoscale level. Alivisators and Mirkin have exploited a DNA recognition linker to form specific nanoparticles combination structures. Stupp and Coworkers nucleated ZnS and CdS in lyotropic liquid crystalline media to make nanowires and nanostructures. Both methods, however, are limited in length scale and offer limited types of inorganic materials with which to work. Therefore, alternative methods of creating well-ordered structures at the nanoscale level are needed.

The present invention is based on the recognition that monodisperse biomaterials that have anisotrophic shape can be a way to build well-ordered structures. The present invention includes methods for building well-ordered nanoparticle layers by using biological selectivity and self-assembly. The nanoparticle layers can be made of Group II-VI semiconductor materials such as CdS, FeS, and ZnS.

One form of the present invention is a method for using self-assembling biological molecules, e.g., bacteriophage, that are genetically engineered to bind to semi-conductor materials and to organize well-ordered structures. These structures may be, e.g., nanoscale arrays of nanoparticles. Using bacteriophage as an example, self-assembling biological materials can be selected for specific binding properties to particular semiconductor surfaces, and thus, the modified bacteriophage and the methods taught herein may be used to create well-ordered structures of the materials selected.

Another form of the present invention is a method of creating nanoparticles that have specific alignment properties. This is accomplished by creating, e.g., an M13 bacteriophage that has specific binding properties, amplifying the bacteriophage to high concentrations using the polymerase chain reaction, and resuspending the phage.

This same method may be used to create bacteriophage that have three liquid crystalline phases, a directional order in the nemetic phase, a twisted nemetic structure in the cholesteric phase, and both directional and positional order in smectic phase. In one aspect the present invention is a method of making a polymer, e.g., a film, comprising the steps of, amplifying a self-assembling biological molecule comprising a portion that binds a specific semiconductor surfaces to high concentrations and contacting one or more semiconductor material precursors with the self-assembling biological molecule to form or direct the formation of a crystal.

Another form of the present invention is method for creating nanoparticles that have differing cholesteric pitches by using, e.g., an M13 bacteriophage that has been selected to bind to semiconductor surfaces and resuspending the phage to various concentrations. Another form of the present invention is a method of preparing a casting film with aligned nanoparticles by using, e.g., genetically engineered M13 bacteriophage and re suspending the bacteriophage.

### BRIEF DESCRIPTION OF THE FIGURES

For a more complete understanding of the features and advantages of the present invention, reference is now made to the detailed description of the invention along with the accompanying figures in which corresponding numerals in the different figures refer to corresponding parts and in which:

FIG. 1 depicts selected random amino acid sequences in accordance with the present invention;

FIG. 2 depicts XPS spectra of structures in accordance with the present invention;

FIG. 3 depicts phage recognition of heterostructures in accordance with the present invention;

FIGS. 4-8 depict specific amino acid sequences in accordance with the present invention;

FIGS. 9(a) and 9(b) depict schematic diagrams of the smectic alignment of M13 phages in accordance with the present invention;

FIGS. 10(a)-10(f) are images of the A7-ZnS suspensions: (a) and (b) POM images, (c) AFM image, (d) SEM image, (e) TEM image and (f) TEM image (with electron diffraction insert); and

FIGS. 11(a)-11(f) are images of the M13 bacteriophage nanoparticle biofilm, (a) photograph of the film, (b) schematic diagram of the film structure, (c) AFM image, (d) SEM image, (e) and (f) TEM images along the x-z and z-y planes.

### DETAILED DESCRIPTION OF THE INVENTION

Although making and using various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention, and do not delimit the scope of the invention.

The inventors have previously shown that peptides can bind to semiconductor materials. These peptides have been further developed into a way of nucleating nanoparticles and directing their self-assembly. The main features of the peptides are their ability to recognize and bind technologically important materials with face specificity, to nucleate size-constrained crystalline semiconductor materials, and to control the crystallographic phase of nucleated nanoparticles. The peptides can also control the aspect ratio of the peptides and therefore, the optical properties.

Briefly, the facility with which biological systems assemble immensely complicated structure on an exceedingly minute scale has motivated a great deal of interest in the desire to identify non-biological systems that can behave in a similar fashion. Of particular value would be methods that could be applied to materials with interesting electronic or optical properties, but natural evolution has not selected for interactions between biomolecules and such materials.

The present invention is based on recognition that biological systems efficiently and accurately assemble nanoscale building blocks into complex and functionally sophisticated structures with high perfection, controlled size and compositional uniformity.

One method of providing a random organic polymer pool is using a Phage-display library, based on a combinatorial library of random peptides containing between 7 and 12 amino acids fused to the pIII coat protein of M13 coliphage, provided different peptides that were reacted with crystalline semiconductor structures. Five copies of the pIII coat protein are located on one end of the phage particle, accounting for 10-16 nm of the particle. The phage-display approach provided a physical linkage between the peptide substrate interaction and the DNA that encodes that interaction. The examples described here used as examples, five different single-crystal semiconductors: GaAs (100), GaAs (111)A, GaAs(111)B, InP(100) and Si(100). These substrates allowed for systematic evaluation of the peptide substrate interactions and confirmation of the general utility of the methodology of the present invention for different crystalline structures.

Protein sequences that successfully bound to the specific crystal were eluted from the surface, amplified by, e.g., a million-fold, and reacted against the substrate under more stringent conditions. This procedure was repeated five times to select the phage in the library with the most specific binding. After, e.g., the third, fourth and fifth rounds of phage selection, crystal-specific phage were isolated and their DNA sequenced. Peptide binding has been identified that is selective for the crystal composition (for example, binding to GaAs but not to Si) and crystalline face (for example, binding to (100) GaAs, but not to (111)B GaAs).

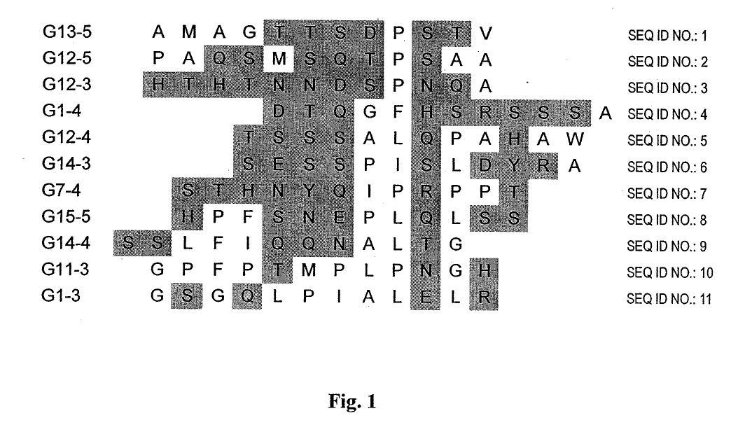

Twenty clones selected from GaAs(100) were analyzed to determine epitope binding domains to the GaAs surface. The partial peptide sequences of the modified pIII or pVIII protein are shown in FIG. 1, revealing similar amino-acid sequences among peptides exposed to GaAs. With increasing number of exposures to a GaAs surface, the number of uncharged polar and Lewis-base functional groups increased. Phage clones from third, fourth and fifth round sequencing contained on average 30%, 40% and 44% polar functional groups, respectively, while the fraction of Lewis-base functional groups increased at the same time from 41% to 48% to 55%. The observed increase in Lewis bases, which should constitute only 34% of the functional groups in random 12-mer peptides from our library, suggests that interactions between Lewis bases on the peptides and Lewis-acid sites on the GaAs surface may mediate the selective binding exhibited by these clones.

The expected structure of the modified 12-mers selected from the library may be an extended conformation, which seems likely for small peptides, making the peptide much longer than the unit cell (5.65 A°) of GaAs. Therefore, only small binding domains would be necessary for the peptide to recognize a GaAs crystal. These short peptide domains, highlighted in FIG. 1, contain serine- and threonine-rich regions in addition to the presence of amine Lewis bases, such as asparagine and glutamine. To determine the exact binding sequence, the surfaces have been screened with shorter libraries, including 7-mer and disulphide constrained 7-mer libraries. Using these shorter libraries that reduce the size and flexibility of the binding domain, fewer peptide-surface interactions are allowed, yielding the expected increase in the strength of interactions between generations of selection.

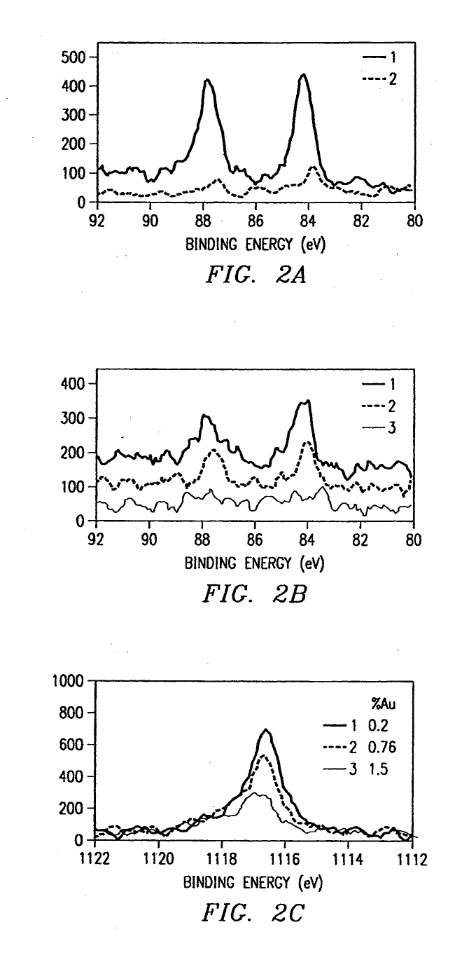

Phage, tagged with streptavidin-labeled 20-nm colloidal gold particles bound to the phage through a biotinylated antibody to the M13 coat protein, were used for quantitative assessment of specific binding. X-ray photoelectron spectroscopy (XPS) elemental composition determination was performed, monitoring the phage substrate interaction through the intensity of the gold 4f-electron signal (FIGS. 2a-c). Without the presence of the G1-3 phage, the antibody and the gold streptavidin did not bind to the GaAs(100) substrate. The gold-streptavidin binding was, therefore, specific to the phage and an indicator of the phage binding to the substrate. Using XPS it was also found that the G1-3 clone isolated from GaAs(100) bound specifically to GaAs (100) but not to Si (100) (see FIG. 2a). In complementary fashion the S1 clone, screened against the (100) Si surface, showed poor binding to the (100) GaAs surface.

Some GaAs clones also bound the surface of InP (100), another zinc-blende structure. The basis of the selective binding, whether it is chemical, structural or electronic, is still under investigation. In addition, the presence of native oxide on the substrate surface may alter the selectivity of peptide binding.

The preferential binding of the G1-3 clone to GaAs(100), over the (111)A (gallium terminated) or (111)B (arsenic terminated) face of GaAs was demonstrated (FIG. 2b, c). The G1-3 clone surface concentration was greater on the (100) surface, which was used for its selection, than on the gallium-rich (111)A or arsenic-rich (111)B surfaces. These different surfaces are known to exhibit different chemical reactivities, and it is not surprising that there is selectivity demonstrated in the phage binding to the various crystal faces. Although the bulk termination of both 111 surfaces give the same geometric structure, the differences between having Ga or As atoms outermost in the surface bilayer become more apparent when comparing surface reconstructions. The composition of the oxides of the various GaAs surfaces is also expected to be different, and this in turn may affect the nature of the peptide binding.

The intensity of Ga 2p electrons against the binding energy from substrates that were exposed to the G1-3 phage clone is plotted in 2c. As expected from the results in FIG. 2b, the Ga 2p intensities observed on the GaAs (100), (111)A and (111)B surfaces are inversely proportional to the gold concentrations. The decrease in Ga 2p intensity on surfaces with higher gold-streptavidin concentrations was due to the increase in surface coverage by the phage. XPS is a surface technique with a sampling depth of approximately 30 angstroms; therefore, as the thickness of the organic layer increases, the signal from the inorganic substrate decreases. This observation was used to confirm that the intensity of gold-streptavidin was indeed due to the presence of phage containing a crystal specific bonding sequence on the surface of GaAs. Binding studies were performed that correlate with the XPS data, where equal numbers of specific phage clones were exposed to various semiconductor substrates with equal surface areas. Wild-type clones (no random peptide insert) did not bind to GaAs (no plaques were detected). For the G1-3 clone, the eluted phage population was 12 times greater from GaAs(100) than from the GaAs(111)A surface.

The G1-3, G12-3 and G7-4 clones bound to GaAs(100) and InP(100) were imaged using atomic force microscopy (AFM). The InP crystal has a zinc-blende structure, isostructural with GaAs, although the In—P bond has greater ionic character than the GaAs bond. The 10-nm width and 900-nm length of the observed phage in AFM matches the dimensions of the M13 phage observed by transmission electron microscopy (TEM), and the gold spheres bound to M13 antibodies were observed bound to the phage (data not shown). The InP surface has a high concentration of phage. These data suggest that many factors are involved in substrate recognition, including atom size, charge, polarity and crystal structure.

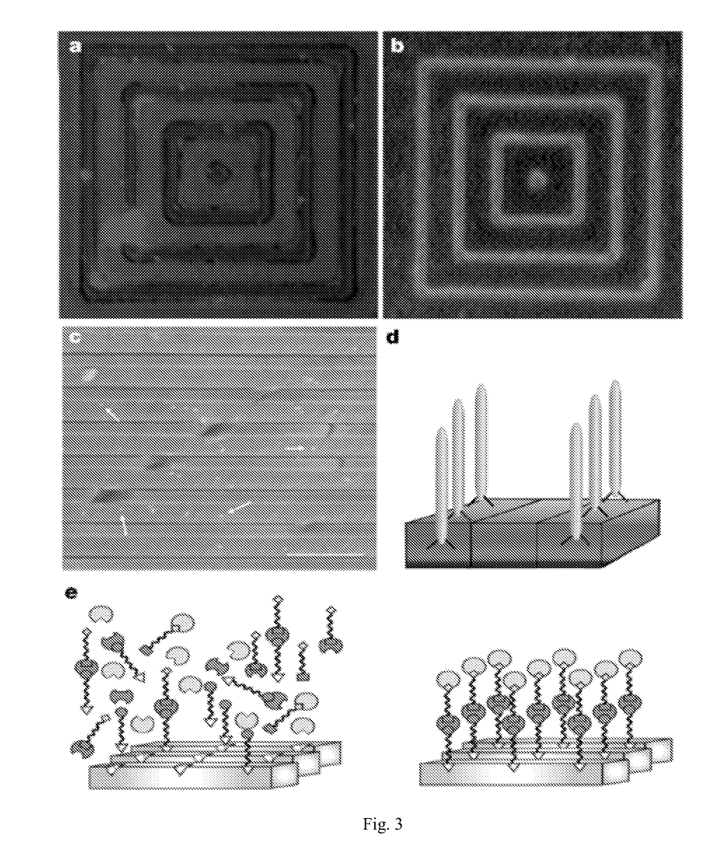

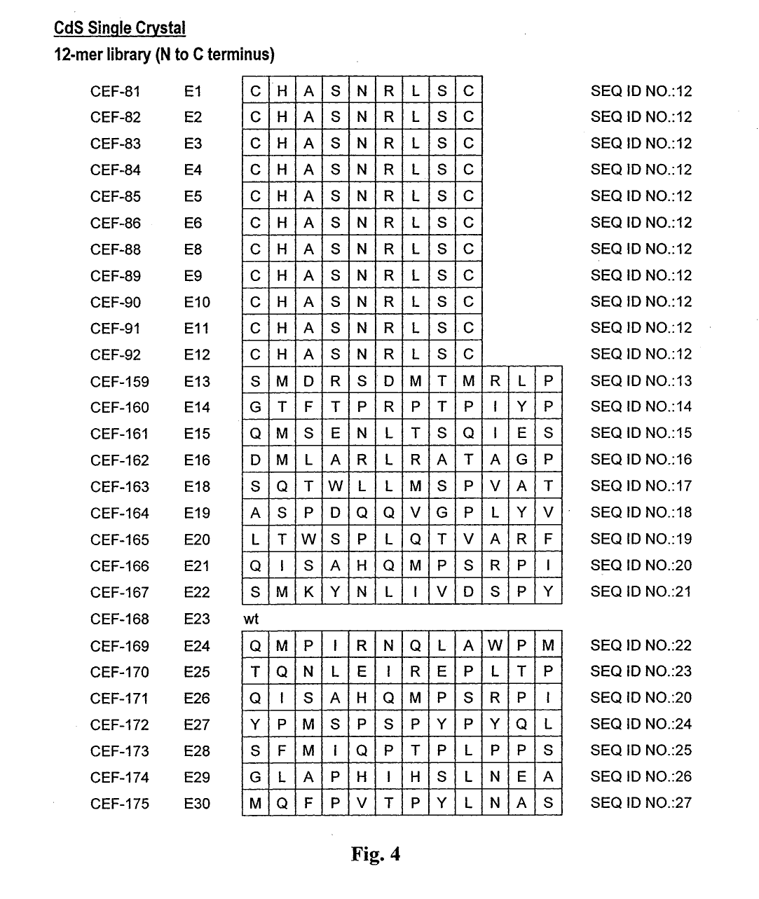

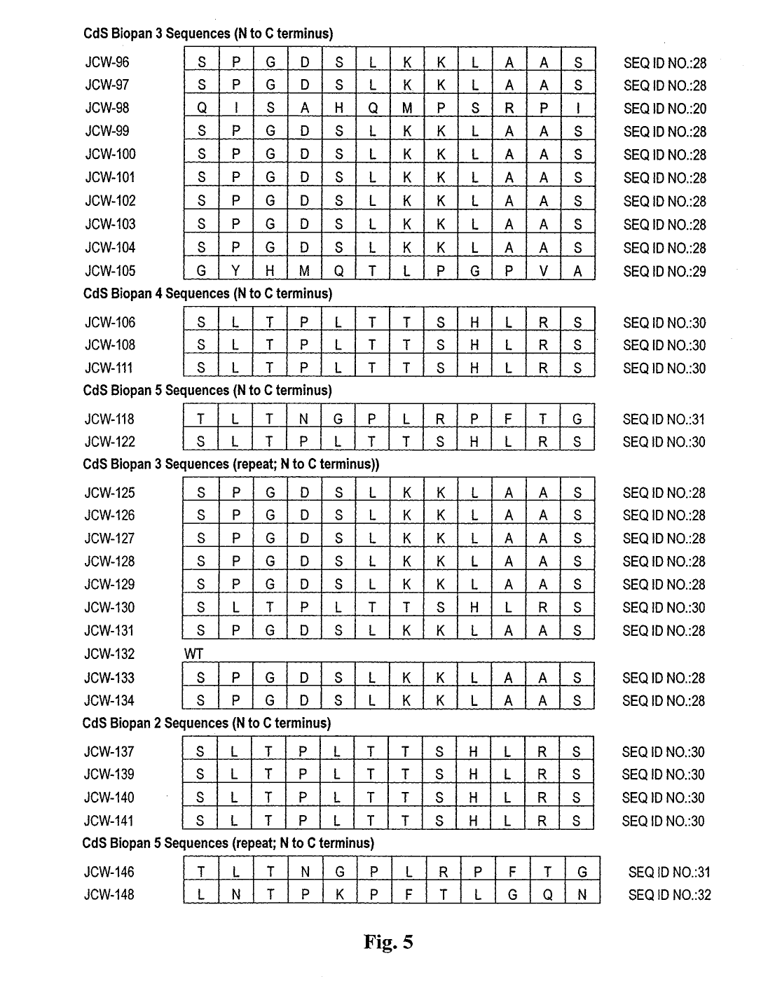

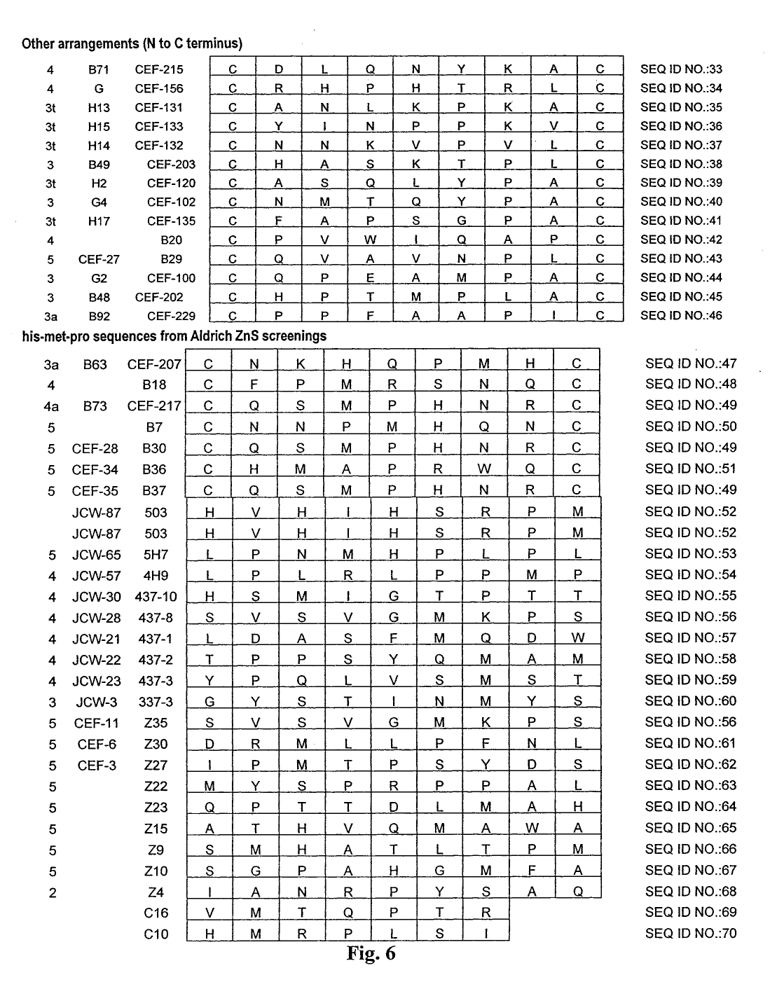

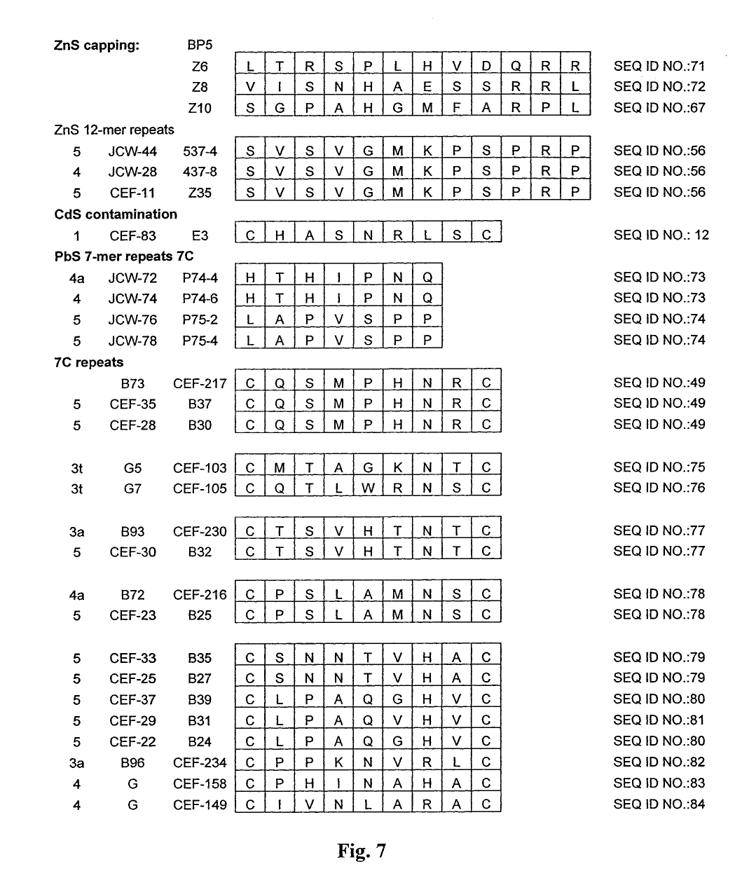

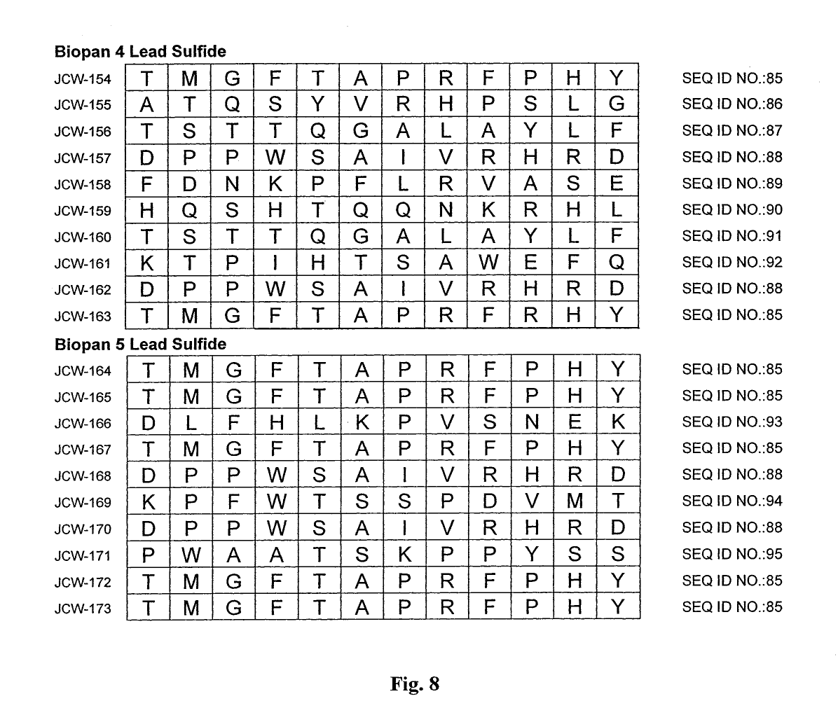

The G1-3 clone (negatively stained) is seen bound to a GaAs crystalline wafer in the TEM image (not shown). The data confirms that binding was directed by the modified pIII protein of G1-3, not through non-specific interactions with the major coat protein. Therefore, peptides of the present invention may be used to direct specific peptide-semiconductor interactions in assembling nanostructures and heterostructures (FIG. 3e).

X-ray fluorescence microscopy was used to demonstrate the preferential attachment of phage to a zinc-blende surface in close proximity to a surface of differing chemical and structural composition. A nested square pattern was etched into a GaAs wafer; this pattern contained 1-μm lines of GaAs, and 4-μm SiO₂ spacing in between each line (FIGS. 3a, 3b). The G12-3 clones were interacted with the GaAs/SiO2 patterned substrate, washed to reduce non-specific binding, and tagged with an immuno-fluorescent probe, tetramethyl rhodamine (TMR). The tagged phage were found as the three red lines and the center dot, in FIG. 3b, corresponding to G12-3 binding only to GaAs. The SiO₂ regions of the pattern remain unbound by phage and are dark in color. This result was not observed on a control that was not exposed to phage, but was exposed to the primary antibody and TMR (FIG. 3a). The same result was obtained using non-phage bound G12-3 peptide.

The GaAs clone G12-3 was observed to be substrate-specific for GaAs over AlGaAs (FIG. 3c). AlAs and GaAs have essentially identical lattice constraints at room temperature, 5.66 A° and 5.65 A°, respectively, and thus ternary alloys of Al×Ga1-×As can be epitaxially grown on GaAs substrates. GaAs and AlGaAs have zinc-blende crystal structures, but the G12-3 clone exhibited selectivity in binding only to GaAs. A multilayer substrate was used, consisting of alternating layers of GaAs and of Al_{0.98}Ga_{0.02}As. The substrate material was cleaved and subsequently reacted with the G12-3 clone.

The G12-3 clones were labeled with 20-nm gold-streptavidin nanoparticles. Examination by scanning electron microscopy (SEM) shows the alternating layers of GaAs and Al_{0.98}Ga_{0.02}As within the heterostructure (FIG. 3c). X-ray elemental analysis of gallium and aluminum was used to map the gold-streptavidin particles exclusively to the GaAs layers of the heterostructure, demonstrating the high degree of binding specificity for chemical composition. In FIG. 3d, a model is depicted for the discrimination of phage for semiconductor heterostructures, as seen in the fluorescence and SEM images (FIGS. 3a-c).

The present invention demonstrates the power use of phage-display libraries to identify, develop and amplify binding between organic peptide sequences and inorganic semiconductor substrates. This peptide recognition and specificity of inorganic crystals has been extended to other substrates, including GaN, ZnS, CdS, Fe₃O₄, Fe₂O₃, CdSe, ZnSe and CaCO₃ using peptide libraries. Bivalent synthetic peptides with two-component recognition (FIG. 3e) are currently being designed; such peptides have the potential to direct nanoparticles to specific locations on a semiconductor structure. These organic and inorganic pairs should provide powerful building blocks for the fabrication of a new generation of complex, sophisticated electronic structures.

### Example I

### Peptide Creation, Isolation, Selection and Characterization

Peptide selection. The phage display or peptide library was contacted with the semiconductor, or other, crystals in Tris-buffered saline (TBS) containing 0.1% TWEEN-20, to reduce phage-phage interactions on the surface. After rocking for 1 h at room temperature, the surfaces were washed with 10 exposures to Tris-buffered saline, pH 7.5, and increasing TWEEN-20 concentrations from 0.1% to 0.5% (v/v). The phage were eluted from the surface by the addition of glycine-HCl (pH 2.2) 10 minute, transferred to a fresh tube and then neutralized with Tris-HCl (pH 9.1). The eluted phage were titered and binding efficiency was compared.

The phage eluted after third-round substrate exposure were mixed with their Escherichia coli ER2537 host and plated on LB XGal/IPTG plates. Since the library phage were derived from the vector M13 mp 19, which carries the lacZα gene, phage plaques were blue in color when plated on media containing Xgal (5-bromo-4-chloro-3-indoyl-β-D-galactoside) and IPTG (isopropyl-β-D-thiogalactoside). Blue/white screening was used to select phage plaques with the random peptide insert. Plaques were picked and DNA sequenced from these plates.

Substrate preparation. Substrate orientations were confirmed by X-ray diffraction, and native oxides were removed by appropriate chemical specific etching. The following etches were tested on GaAs and InP surfaces: NH₄OH:H₂O 1:10, HCl:H₂O 1:10, H₃PO₄:H₂O₂:H₂O 3:1:50 at 1 minute and 10 minute etch times. The best element ratio and least oxide formation (using XPS) for GaAs and InP etched surfaces was achieved using HCl:H₂O for 1 minute followed by a deionized water rinse for 1 minute. However, since an ammonium hydroxide etch was used for GaAs in the initial screening of the library, this etch was used for all other GaAs substrate examples. Si(100) wafers were etched in a solution of HF:H₂O 1:40 for one minute, followed by a deionized water rinse. All surfaces were taken directly from the rinse solution and immediately introduced to the phage library. Surfaces of control substrates, not exposed to phage, were characterized and mapped for effectiveness of the etching process and morphology of surfaces by AFM and XPS.

Multilayer substrates of GaAs and of Al_{0.98}Ga_{0.02}As were grown by molecular beam epitaxy onto (100) GaAs. The epitaxially grown layers were Si-doped (n-type) at a level of 5×10¹⁷ cm⁻³.

Antibody and Gold Labeling. For the XPS, SEM and AFM examples, substrates were exposed to phage for 1 h in Tris-buffered saline then introduced to an anti-fd bacteriophage-biotin conjugate, an antibody to the pIII protein of fd phage, (1:500 in phosphate buffer, Sigma) for 30 minute and then rinsed in phosphate buffer. A streptavidin/20-nm colloidal gold label (1:200 in phosphate buffered saline (PBS), Sigma) was attached to the biotin-conjugated phage through a biotin-streptavidin interaction; the surfaces were exposed to the label for 30 minutes and then rinsed several times with PBS.

X-ray Photoelectron Spectroscopy (XPS). The following controls were done for the XPS examples to ensure that the gold signal seen in XPS was from gold bound to the phage and not non-specific antibody interaction with the GaAs surface. The prepared (100) GaAs surface was exposed to (1) antibody and the streptavidin-gold label, but without phage, (2) G1-3 phage and streptavidin-gold label, but without the antibody, and (3) streptavidin-gold label, without either G1-3 phage or antibody.

The XPS instrument used was a Physical Electronics Phi ESCA 5700 with an aluminum anode producing monochromatic 1,487-eV X-rays. All samples were introduced to the chamber immediately after gold-tagging the phage (as described above) to limit oxidation of the GaAs surfaces, and then pumped overnight at high vacuum to reduce sample outgassing in the XPS chamber.

Atomic Force Microscopy (AFM). The AFM used was a Digital Instruments Bioscope mounted on a Zeiss Axiovert 100s-2tv, operating in tip scanning mode with a G scanner. The images were taken in air using tapping mode. The AFM probes were etched silicon with 125-mm cantilevers and spring constants of 20±100 Nm−1 driven near their resonant frequency of 200±400 kHz. Scan rates were of the order of 1±5 mms−1. Images were leveled using a first-order plane to remove sample tilt.

Transmission Electron Microscopy (TEM). TEM images were taken using a Philips EM208 at 60 kV. The G1-3 phage (diluted 1:100 in TBS) were incubated with GaAs pieces (500 mm) for 30 minute, centrifuged to separate particles from unbound phage, rinsed with TBS, and resuspended in TBS. Samples were stained with 2% uranyl acetate.

Scanning Electron Microscopy (SEM). The G12-3 phage (diluted 1:100 in TBS) were incubated with a freshly cleaved hetero-structure surface for 30 minute and rinsed with TBS. The G12-3 phage were tagged with 20-nm colloidal gold. SEM and elemental mapping images were collected using the Norian detection system mounted on a Hitachi 4700 field emission scanning electron microscope at 5 kV.

### Example II

### Biofilms

The present inventors have recognized that organic-inorganic hybrid materials offer new routes for novel materials and devices. Size controlled nanostructures give optically and electrically tunable properties of semiconductor materials and organic additives modify the inorganic morphology, phase, and nucleation direction. The monodispersed nature of biological materials makes the system compatible for highly ordered smectic-ordering structure. Using the methods of the present invention, highly ordered nanometer scale as well as multi-length scale alignment of II-VI semiconductor material using genetically engineered, self-assembling, biological molecules, e.g., M13 bacteriophage that have a recognition moiety of specific semiconductor surfaces were created.

Using the compositions and methods of the present invention nano- and multi-length scale alignment of semiconductor materials was achieved using the recognition and self-ordering system described herein. The recognition and self-ordering of semiconductors may be used to enhance micro fabrication of electronic devices that surpass current photolithographic capabilities. Application of these materials include: optoelectronic devices such as light emitting displays, optical detectors and lasers; fast interconnects; and nano-meter scale computer components and biological sensors. Other uses of the biofilms created using the present invention include well-ordered liquid crystal displays and organic-inorganic display technology.

The films, fibers and other structures may even include high density sensors for detection of small molecules including biological toxins. Other uses include optical coatings and optical switches. Optionally, scaffoldings for medical implants or even bone implants; may be constructed using one or more of the materials disclosed herein, in single or multiple layers or even in striations or combinations of any of these, as will be apparent to those of skill in the art. Other uses for the present invention include electrical and magnetic interfaces, or even the organization of 3D electronic nanostructures for high density storage, e.g., for use in quantum computing. Alternatively, high density and stable storage of viruses for medical application that can be reconstituted, e.g., biologically compatible vaccines, adjuvants and vaccine containers may be created with the films and or matrices created with the present invention. Information storage based on quantum dot patterns for identification, e.g., department of defense friend or foe identification in fabric of armor or coding. The present nanofibers may even be used to code and identify money.

Building well-ordered, well-controlled, two and three dimensional structure at the nanolength scale is the major goal of building next generation optical, electronic and magnetic materials and devices. Current methods of making specific nanoparticles are limited in terms of both length scale and the types of materials. The present invention exploits the properties of self-assembling organic or biological molecules or particles, e.g., M13 bacteriophage to expand the alignment, size, and scale of the nanoparticles as well as the range of semiconductor materials that can be used.

The present inventors have recognized that monodisperse biomaterials having anisotrophic shapes are an alternative way to build well-ordered structures. Nano and multi-length scale alignment of II-VI semiconductor material was accomplished using genetically engineered M13 bacteriophage that possess a recognition moiety (a peptide or amino acid oligomer) for specific semiconductor surfaces.

Seth and coworkers have characterized Fd virus smectic ordering structures that have both a positional and directional order. The smectic structure of Fd virus has potential application in both multi-scale and nanoscale ordering of structures to build 2-dimensional and 3-dimensional alignment of nanoparticles. Bacteriophage M13 was used because it can be genetically modified, has been successfully selected to have a shape identical to the Fd virus, and has specific binding affinities for II-VI semiconductor surfaces. Therefore, M13 is an ideal source for smectic structure that can serve in multi-scale and nanoscale ordering of nanoparticles.

The present inventors have used combinatorial screening methods to find M13 bacteriophage containing peptide inserts that are capable of binding to semiconductor surfaces. These semiconductor surfaces included materials such as zinc sulfide, cadmium sulfide and iron sulfide. Using the techniques of molecular biology, bacteriophage combinatorial library clones that bind specific semi-conductor materials and material surfaces were cloned and amplified up to concentrations high enough for liquid crystal formation.

The filamentous bacteriophage, Fd, has a long rod shape (length: 880 nm; diameter: 6.6 nm) and monodisperse molecular weight (molecular weight: 1.64×10⁷). These properties result in the bacteriophage's lyotropic liquid crystalline behavior in highly concentrated solutions. The anisotrophic shape of bacteriophage was exploited as a method to build well-ordered nanoparticle layers by use of biological selectivity and self-assembly. Monodisperse bacteriophage were prepared through standard amplification methods. In the present invention, M13, a similar filamentous bacteriophage, was genetically modified to bind nanoparticles such as zinc sulfide, cadmium sulfide and iron sulfide.

Mesoscale ordering of bacteriophage has been demonstrated to form nanoscale arrays of nanoparticles. These nanoparticles are further organized into micron domains and into centimeter length scales. The semiconductor nanoparticles show quantum confinement effects, and can be synthesized and ordered within the liquid crystal.

Bacteriophage M13 suspension containing specific peptide inserts were made and characterized using Atomic Force Microscopy (ATM), Transmission Electron Microscopy (TEM) and Scanning Electron Microscopy (SEM). Uniform 2D and 3D ordering of nanoparticles was observed throughout the samples.

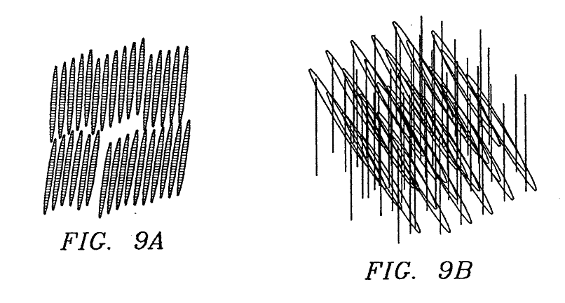

Atomic Force Microscopy (AFM). The AFM used was a Digital Instruments Bioscope mounted on a Zeiss Axiovert 100s-2tv, operating in tip scanning mode with a G scanner. The images were taken in air using tapping mode. The AFM probes were etched silicon with 125-mm cantilevers and spring constants of 20±100 Nm−1 driven near their resonant frequency of 200±400 kHz. Scan rates were of the order of 1±5 mms−1. Images were leveled using a first-order plane to remove sample tilt. FIGS. 9(a) and 9(b) are schematic diagrams of the smetic alignment of M13 phages observed using AFM (data not shown).

Transmission Electron Microscopy (TEM). TEM images were taken using a Philips EM208 at 60 kV. The G1-3 phage (diluted 1:100 in TBS) were incubated with semiconductor material for 30 minute, centrifuged to separate particles from unbound phage, rinsed with TBS, and resuspended in TBS. Samples were stained with 2% uranyl acetate.

Scanning Electron Microscopy (SEM). The phage (diluted 1:100 in TBS) were incubated with a freshly cleaved hetero-structure surface for 30 minute and rinsed with TBS. The G12-3 phage were tagged with 20-nm colloidal gold. SEM and elemental mapping images were collected using the Norian detection system mounted on a Hitachi 4700 field emission scanning electron microscope at 5 kV.

Genetically engineered M13 bacteriophage that had specific binding properties to semiconductor surfaces was amplified and purified using standard molecular biological techniques. 3.2 ml of bacteriophage suspension (concentration: ˜10⁷ phages/ul) and 4 ml of overnight culture were added to 400 ml LB medium for mass amplification. After amplification, ˜30 mg of pellet was precipitated. The suspensions were prepared by adding Na₂S solutions to ZnCl₂ doped A7 phage suspensions at room temperature. The highest concentration of A7-phage suspension was prepared by adding 20 ul of 1 mM ZnCl₂ and Na₂S solutions, respectively into the mg of phage pellet. The concentration was measured using extinction coefficient of 3.84 mg/ml at 269 nm.

As the concentration of the isotropic suspension is increased, nemetic phase that has directional order, cholesteric phase that has twisted nemetic structure, and smectic phase that has directional and positional orders as well, are observed. These phases had been observed in Fd viruses that did not have nanoparticles.

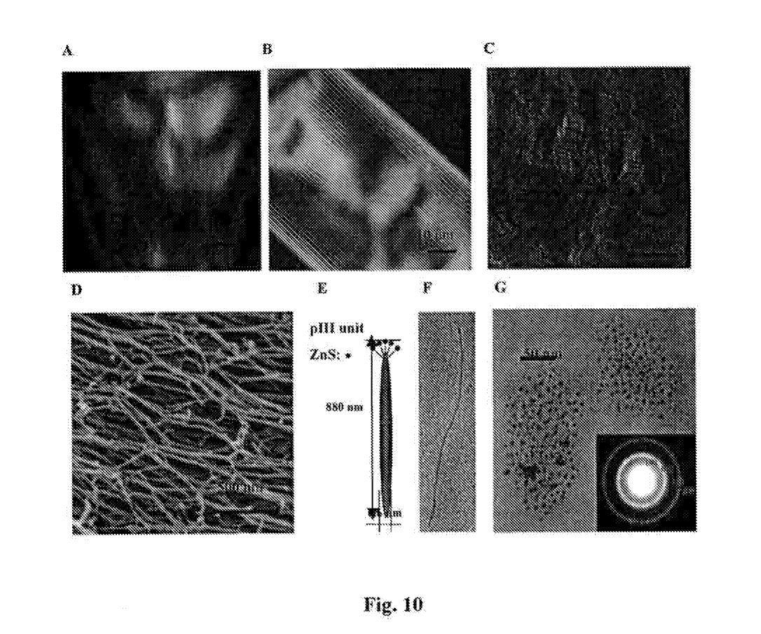

Polarized optical microscopy: M13 phage suspensions were characterized by polarized optical microscope. Each suspension was filled to glass capillary tube of 0.7 mm diameter. The highly concentrated suspension (127 mg/ml) exhibited iridescent color [5] under the paralleled polarized light and showed smectic texture under the cross-polarized light as FIG. 10(a). The cholesteric pitches, FIG. 10(b) can be controlled by varying the concentration of suspension as Table 1. The pitch length was measured and the micrographs were taken after 24 hours later from the preparation of samples.

TABLE 1
Cholesteric pitch and concentration relationship

| Concentration | Pitch length |
|---|---|
| (mg/ml) | (um) |
| 76.30 | 31.9 |
| 71.22 | 51.6 |
| 56.38 | 84.8 |
| 50.52 | 101.9 |
| 43.16 | 163.7 |
| 37.04 | 176.1 |
| 27.54 | 259.7 |

Atomic Force Microscope (AFM) observation: For AFM observation, 5 ul of M13 suspension (concentration: 30 mg/ml) of M13 bacteriophage suspension was dried for 24 hours on the 8 mm×8 mm mica substrate that was silated by 3-amino propyl triethyl silane for 4 hours in the dessicator. Images were taken in air using tapping mode. Self-assembled ordering structures were observed due to the anisotropic shape of M13 bacteriophage, 880 nm in length and 6.6 nm in width. In FIG. 10 (c) M13 phage lie in the plane of the photo and form smectic alignment.

Scanning electron microscope (SEM) observation: For SEM observation, the critical point drying samples of bacteriophage and ZnS nanoparticles smectic suspension (concentration of bacteriophage suspension 127 mg/ml) were prepared. In FIG. 10(d), nanoparticles rich areas and bacteriophage rich areas were observed. The length of the separation between nanoparticles and bacteriophage correspond to the length of bacteriophage. The ZnS wurzite crystal structure was confirmed by electron diffraction pattern using dilution sample of the smectic suspension with TEM.

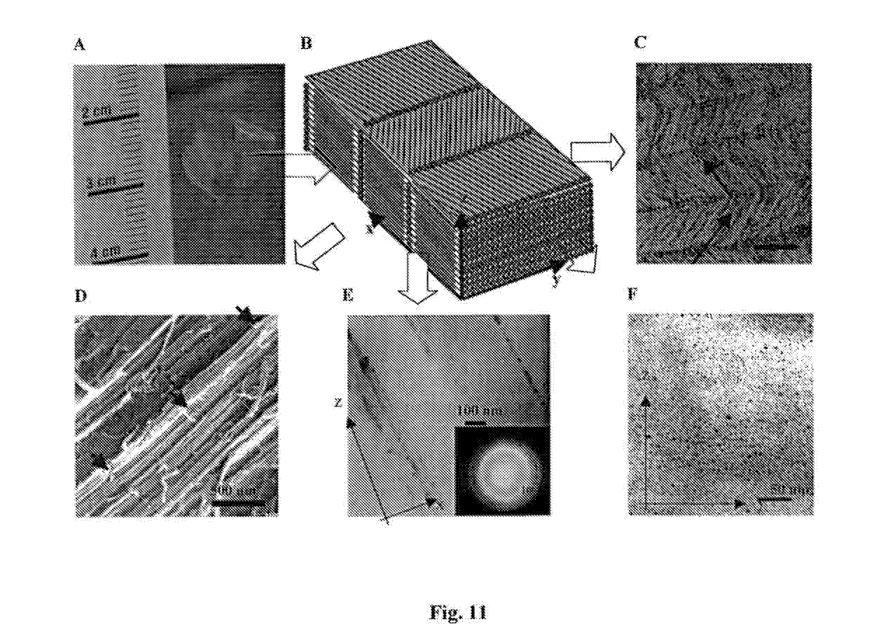

Preparation of the biofilm: Bacteriophage Pellets were suspended with 400 ul of Tris-buffered saline (TBS, pH 7.5) and 200 ul of 1 mM ZnCl₂ to which 1 mM Na₂S was added. After rocking for 24 hours at room temperature, the suspension which was contained in a 1 ml eppendorff tube, was slowly dried in a dessicator for one week. A semi-transparent film ˜15 um thick was formed on the inside of the tube. This film, FIG. 11(a), was carefully taken using a tweezers.

SEM observation of biofilm: Nanoscale bacteriophage alignment of the A7-ZnS film were observed using SEM. In order to carry out SEM analysis the film was cut then coated via vacuum deposition with 2 nm of chromium in an argon atmosphere. Highly close-packed structures, FIG. 11(d) were observed throughout the sample. The average length of individual phage, 895 nm is reasonable analogous to that of phage, 880 nm. The film showed the smectic like A or C like lamellar morphologies that exhibited periodicity between the nanoparticle and bacteriophage layers. The length of periodicity corresponded to that of the bacteriophage. The average size of nanoparticle is −20 nm analogous to the TEM observation of individual particles.

TEM observation of biofilm: ZnS nanoparticle alignment was investigated using TEM. The film was embedded in epoxy resin (LR white) for one day and polymerized by adding 10 ul of accelerator. After curing, the resin was thin sectioned using a Leica Ultramicrotome. These ˜50 nm sections were floated on distilled water, and picked up on blank gold grids. Parallel-aligned nanoparticles in a low, which corresponded to x-z plane in the schematic diagram, were observed, FIG. 11(e). Since each bacteriophage had 5 copies of the A7 moieties, each A7 recognize one nanoparticle (2˜3 nm size) and aligned approximately 20 nm in a width and extended to more than two micrometers in length. The two micrometers by 20 nm bands formed in parallel each band separated by ˜700 nm. This discrepancy may come from the tilted smectic alignment of the phage layers with respect to observation in the TEM, which is reported by Marvin group. A y-z axis like nanoparticle layer plane was also observed like FIG. 3 (f). The SAED patterns of the aligned particles showed that the ZnS particles have the wurzite hexagonal structure.

AFM observation of biofilm: The surface orientation of the viral film was investigated using AFM. In FIG. 11. (c), the phage were shown to have formed an parallel aligned herringbone pattern that have almost right angle between the adjacent director normal (bacteriophage axis) on most of surface that is named as smectic O. The film showed long range ordering of normal director that is persistent to the tens of micrometers. In some of areas where two domain layers meet each other, two or three multi-length scale of bacteriophage aligned paralleled and persistent to the smectic C ordering structure.

Nano and multi-length scale alignment of semiconductor materials using the recognition and as well as self-ordering system enhances the future microfabrication of electronic devices. These devices have the potential to surpass current photolithographic capabilities. Other potential applications of these materials include optoelectronic devices such as light-emitting displays, optical detectors, and lasers, fast interconnects, nano-meter scale computer component and biological sensors.

Although making and using various embodiments of the present invention are discussed in detail below, it will be appreciated that the present invention provides many applicable inventive concepts that can be embodied in a wide variety of specific contexts. The specific embodiments discussed herein are merely illustrative of specific ways to make and use the invention, and do not delimit the scope of the invention.

What is claimed is:

1. A method for forming a film comprising:
(i) preparing a composition comprising at least one self-assembling bacteriophage with at least one synthetic peptide or protein expressed on the surface of said bacteriophage, wherein the at least one peptide or protein contains sequences of amino acids that are selective for at least one type of inorganic nanocrystals,
(ii) contacting said composition with a solution comprising an inorganic material that is a precursor for said at least one type of inorganic nanocrystal, and
(iii) concentrating the mixture formed in (ii) to produce a film in which the at least one peptide or protein has nucleated and bound to the at least one type of inorganic nanocrystals and in which the bacteriophage have self-assembled into the film.

2. The method of claim 1, wherein the at least one type of inorganic nanocrystals form at least one ordered layer in the film.

3. The method of claim 1, wherein the at least one peptide or protein can further nucleate and bind nanocrystals.

4. The method of claim 1, wherein the peptides or proteins are bound to the at least one type of inorganic nanocrystals which are organized into layer domains.

5. The method of claim 1, wherein the at least one peptide or protein is bound to the at least one type of inorganic nanocrystals which are organized into centimeter length scales.

6. The method of claim 1, wherein the at least one peptide is between about 7 to 15 amino acids in length.

7. The method of claim 1, wherein the peptides or proteins are part of p3 bacteriophages.

8. The method of claim 1, wherein the peptides or proteins are part of p8 bacteriophages.

9. The method of claim 1, wherein the at least one type of inorganic nanocrystals are single crystals.

10. The method of claim 1, wherein the at least one type of inorganic nanocrystals are heterostructured.

11. The method of claim 1, wherein the at least one type of inorganic nanocrystals are semiconductor nanocrystals.

12. The method of claim 1, wherein the at least one type of inorganic nanocrystals comprise zinc sulfide, gallium arsenide, indium phosphate, cadmium sulfide, aluminum arsenide, aluminum stibinide, or silicon.

13. The method of claim 1, wherein the at least one type of inorganic nanocrystals are CdS, FeS, ZnS, GaN, Fe3O4, Fe2O3, CdSe, ZnSe, or calcium carbonate.

14. The method of claim 1, wherein the at least one type of inorganic nanocrystals are silica or calcium carbonate.

15. The method of claim 1, wherein the composition is in liquid crystalline film form.


SEQUENCE LISTING (95 sequences)

SEQ ID NO: 1 - protein, 12 aa, artificial sequence
  peptide recognition sequence retrieved from phage biopanning
AMAGTTSDPSTV

SEQ ID NO: 2 - protein, 12 aa, artificial sequence
  peptide recognition sequence retrieved from phage biopanning
AASPTQSMSQAP

SEQ ID NO: 3 - protein, 12 aa, artificial sequence
  peptide recognition sequence retrieved from phage biopanning
HTHTNNDSPNQA

SEQ ID NO: 4 - protein, 12 aa, artificial sequence
  peptide recognition sequence retrieved from phage biopanning
DTQGFHSRSSSA

SEQ ID NO: 5 - protein, 12 aa, artificial sequence
  peptide recognition sequence retrieved from phage biopanning
TSSSALQPAHAW

SEQ ID NO: 6 - protein, 12 aa, artificial sequence
  peptide recognition sequence retrieved from phage biopanning
SESSPISLDYRA

SEQ ID NO: 7 - protein, 12 aa, artificial sequence
  peptide recognition sequence retrieved from phage biopanning
STHNYQIPRPPT

SEQ ID NO: 8 - protein, 12 aa, artificial sequence
  peptide recognition sequence retrieved from phage biopanning
HPFSNEPLQLSS

SEQ ID NO: 9 - protein, 12 aa, artificial sequence
  peptide recognition sequence retrieved from phage biopanning
GTLANQQIFLSS

SEQ ID NO: 10 - protein, 12 aa, artificial sequence
  peptide recognition sequence retrieved from phage biopanning
HGNPLPMTPFPG

SEQ ID NO: 11 - protein, 12 aa, artificial sequence
  peptide recognition sequence retrieved from phage biopanning
RLELAIPLQGSG

SEQ ID NO: 12 - protein, 9 aa, artificial sequence
  peptide recognition sequence retrieved from phage biopanning
CHASNRLSC

SEQ ID NO: 13 - protein, 12 aa, artificial sequence
  peptide recognition sequence retrieved from phage biopanning
SMDRSDMTMRLP

SEQ ID NO: 14 - protein, 12 aa, artificial sequence
  peptide recognition sequence retrieved from phage biopanning
GTFTPRPTPIYP

SEQ ID NO: 15 - protein, 12 aa, artificial sequence
  peptide recognition sequence retrieved from phage biopanning
QMSENLTSQIES

SEQ ID NO: 16 - protein, 12 aa, artificial sequence
  peptide recognition sequence retrieved from phage biopanning
DMLARLRATAGP

SEQ ID NO: 17 - protein, 12 aa, artificial sequence
  peptide recognition sequence retrieved from phage biopanning
SQTWLLMSPVAT

SEQ ID NO: 18 - protein, 12 aa, artificial sequence
  peptide recognition sequence retrieved from phage biopanning
ASPDQQVGPLYV

SEQ ID NO: 19 - protein, 12 aa, artificial sequence
  peptide recognition sequence retrieved from phage biopanning
LTWSPLQTVARF

SEQ ID NO: 20 - protein, 12 aa, artificial sequence
  peptide recognition sequence retrieved from phage biopanning
QISAHQMPSRPI

SEQ ID NO: 21 - protein, 12 aa, artificial sequence
  peptide recognition sequence retrieved from phage biopanning
SMKYNLIVDSPY

SEQ ID NO: 22 - protein, 12 aa, artificial sequence
  peptide recognition sequence retrieved from phage biopanning
QMPIRNQLAWPM

SEQ ID NO: 23 - protein, 12 aa, artificial sequence
  peptide recognition sequence retrieved from phage biopanning
TQNLEIREPLTP

SEQ ID NO: 24 - protein, 12 aa, artificial sequence
  peptide recognition sequence retrieved from phage biopanning
YPMSPSPYPYQL

SEQ ID NO: 25 - protein, 12 aa, artificial sequence
  peptide recognition sequence retrieved from phage biopanning
SFMIQPTPLPPS

SEQ ID NO: 26 - protein, 12 aa, artificial sequence
  peptide recognition sequence retrieved from phage biopanning
GLAPHIHSLNEA

SEQ ID NO: 27 - protein, 12 aa, artificial sequence
  peptide recognition sequence retrieved from phage biopanning
MQFPVTPYLNAS

SEQ ID NO: 28 - protein, 12 aa, artificial sequence
  peptide recognition sequence retrieved from phage biopanning
SPGDSLKKLAAS

SEQ ID NO: 29 - protein, 12 aa, artificial sequence
  peptide recognition sequence retrieved from phage biopanning
GYHMQTLPGPVA

SEQ ID NO: 30 - protein, 12 aa, artificial sequence
  peptide recognition sequence retrieved from phage biopanning
SLTPLTTSHLRS

SEQ ID NO: 31 - protein, 12 aa, artificial sequence
  peptide recognition sequence retrieved from phage biopanning
TLTNGPLRPFTG

SEQ ID NO: 32 - protein, 12 aa, artificial sequence
  peptide recognition sequence retrieved from phage biopanning
LNTPKPFTLGQN

SEQ ID NO: 33 - protein, 9 aa, artificial sequence
  peptide recognition sequence retrieved from phage biopanning
CDLQNYKAC

SEQ ID NO: 34 - protein, 9 aa, artificial sequence
  peptide recognition sequence retrieved from phage biopanning
CRHPHTRLC

SEQ ID NO: 35 - protein, 9 aa, artificial sequence
  peptide recognition sequence retrieved from phage biopanning
CANLKPKAC

SEQ ID NO: 36 - protein, 9 aa, artificial sequence
  peptide recognition sequence retrieved from phage biopanning
CYINPPKVC

SEQ ID NO: 37 - protein, 9 aa, artificial sequence
  peptide recognition sequence retrieved from phage biopanning
CNNKVPVLC

SEQ ID NO: 38 - protein, 9 aa, artificial sequence
  peptide recognition sequence retrieved from phage biopanning
CHASKTPLC

SEQ ID NO: 39 - protein, 9 aa, artificial sequence
  peptide recognition sequence retrieved from phage biopanning
CASQLYPAC

SEQ ID NO: 40 - protein, 9 aa, artificial sequence
  peptide recognition sequence retrieved from phage biopanning
CNMTQYPAC

SEQ ID NO: 41 - protein, 9 aa, artificial sequence
  peptide recognition sequence retrieved from phage biopanning
CFAPSGPAC

SEQ ID NO: 42 - protein, 9 aa, artificial sequence
  peptide recognition sequence retrieved from phage biopanning
CPVWIQAPC

SEQ ID NO: 43 - protein, 9 aa, artificial sequence
  peptide recognition sequence retrieved from phage biopanning
CQVAVNPLC

SEQ ID NO: 44 - protein, 9 aa, artificial sequence
  peptide recognition sequence retrieved from phage biopanning
CQPEAMPAC

SEQ ID NO: 45 - protein, 9 aa, artificial sequence
  peptide recognition sequence retrieved from phage biopanning
CHPTMPLAC

SEQ ID NO: 46 - protein, 9 aa, artificial sequence
  peptide recognition sequence retrieved from phage biopanning
CPPFAAPIC

SEQ ID NO: 47 - protein, 9 aa, artificial sequence
  peptide recognition sequence retrieved from phage biopanning
CNKHQPMHC

SEQ ID NO: 48 - protein, 9 aa, artificial sequence
  peptide recognition sequence retrieved from phage biopanning
CFPMRSNQC

SEQ ID NO: 49 - protein, 9 aa, artificial sequence
  peptide recognition sequence retrieved from phage biopanning
CQSMPHNRC

SEQ ID NO: 50 - protein, 9 aa, artificial sequence
  peptide recognition sequence retrieved from phage biopanning
CNNPMHQNC

SEQ ID NO: 51 - protein, 9 aa, artificial sequence
  peptide recognition sequence retrieved from phage biopanning
CHMAPRWQC

SEQ ID NO: 52 - protein, 9 aa, artificial sequence
  peptide recognition sequence retrieved from phage biopanning
HVHIHSRPM

SEQ ID NO: 53 - protein, 9 aa, artificial sequence
  peptide recognition sequence retrieved from phage biopanning
LPNMHPLPL

SEQ ID NO: 54 - protein, 9 aa, artificial sequence
  peptide recognition sequence retrieved from phage biopanning
LPLRLPPMP

SEQ ID NO: 55 - protein, 9 aa, artificial sequence
  peptide recognition sequence retrieved from phage biopanning
HSMIGTPTT

SEQ ID NO: 56 - protein, 9 aa, artificial sequence
  peptide recognition sequence retrieved from phage biopanning
SVSVGMKPS

SEQ ID NO: 57 - protein, 9 aa, artificial sequence
  peptide recognition sequence retrieved from phage biopanning
LDASFMQDW

SEQ ID NO: 58 - protein, 9 aa, artificial sequence
  peptide recognition sequence retrieved from phage biopanning
TPPSYQMAM

SEQ ID NO: 59 - protein, 9 aa, artificial sequence
  peptide recognition sequence retrieved from phage biopanning
YPQLVSMST

SEQ ID NO: 60 - protein, 9 aa, artificial sequence
  peptide recognition sequence retrieved from phage biopanning
GYSTINMYS

SEQ ID NO: 61 - protein, 9 aa, artificial sequence
  peptide recognition sequence retrieved from phage biopanning
DRMLLPFNL

SEQ ID NO: 62 - protein, 9 aa, artificial sequence
  peptide recognition sequence retrieved from phage biopanning
IPMTPSYDS

SEQ ID NO: 63 - protein, 9 aa, artificial sequence
  peptide recognition sequence retrieved from phage biopanning
MYSPRPPAL

SEQ ID NO: 64 - protein, 9 aa, artificial sequence
  peptide recognition sequence retrieved from phage biopanning
QPTTDLMAH

SEQ ID NO: 65 - protein, 9 aa, artificial sequence
  peptide recognition sequence retrieved from phage biopanning
ATHVQMAWA

SEQ ID NO: 66 - protein, 9 aa, artificial sequence
  peptide recognition sequence retrieved from phage biopanning
SMHATLTPM

SEQ ID NO: 67 - protein, 9 aa, artificial sequence
  peptide recognition sequence retrieved from phage biopanning
SGPAHGMFA

SEQ ID NO: 68 - protein, 9 aa, artificial sequence
  peptide recognition sequence retrieved from phage biopanning
IANRPYSAQ

SEQ ID NO: 69 - protein, 7 aa, artificial sequence
  peptide recognition sequence retrieved from phage biopanning
VMTQPTR

SEQ ID NO: 70 - protein, 7 aa, artificial sequence
  peptide recognition sequence retrieved from phage biopanning
HMRPLSI

SEQ ID NO: 71 - protein, 12 aa, artificial sequence
  peptide recognition sequence retrieved from phage biopanning
LTRSPLHVDQRR

SEQ ID NO: 72 - protein, 12 aa, artificial sequence
  peptide recognition sequence retrieved from phage biopanning
VISNHAESSRRL

SEQ ID NO: 73 - protein, 7 aa, artificial sequence
  peptide recognition sequence retrieved from phage biopanning
HTHIPNQ

SEQ ID NO: 74 - protein, 7 aa, artificial sequence
  peptide recognition sequence retrieved from phage biopanning
LAPVSPP

SEQ ID NO: 75 - protein, 9 aa, artificial sequence
  peptide recognition sequence retrieved from phage biopanning
CMTAGKNTC

SEQ ID NO: 76 - protein, 9 aa, artificial sequence
  peptide recognition sequence retrieved from phage biopanning
CQTLWRNSC

SEQ ID NO: 77 - protein, 9 aa, artificial sequence
  peptide recognition sequence retrieved from phage biopanning
CTSVHTNTC

SEQ ID NO: 78 - protein, 9 aa, artificial sequence
  peptide recognition sequence retrieved from phage biopanning
CPSLAMNSC

SEQ ID NO: 79 - protein, 9 aa, artificial sequence
  peptide recognition sequence retrieved from phage biopanning
CSNNTVHAC

SEQ ID NO: 80 - protein, 9 aa, artificial sequence
  peptide recognition sequence retrieved from phage biopanning
CLPAQGHVC

SEQ ID NO: 81 - protein, 9 aa, artificial sequence
  peptide recognition sequence retrieved from phage biopanning
CLPAQVHVC

SEQ ID NO: 82 - protein, 9 aa, artificial sequence
  peptide recognition sequence retrieved from phage biopanning
CPPKNVRLC

SEQ ID NO: 83 - protein, 9 aa, artificial sequence
  peptide recognition sequence retrieved from phage biopanning
CPHINAHAC

SEQ ID NO: 84 - protein, 9 aa, artificial sequence
  peptide recognition sequence retrieved from phage biopanning
CIVNLARAC

SEQ ID NO: 85 - protein, 12 aa, artificial sequence
  peptide recognition sequence retrieved from phage biopanning
TMGFTAPRFPHY

SEQ ID NO: 86 - protein, 12 aa, artificial sequence
  peptide recognition sequence retrieved from phage biopanning
ATQSYVRHPSLG

SEQ ID NO: 87 - protein, 12 aa, artificial sequence
  peptide recognition sequence retrieved from phage biopanning
TSTTQGALAYLF

SEQ ID NO: 88 - protein, 12 aa, artificial sequence
  peptide recognition sequence retrieved from phage biopanning
DPPWSAIVRHRD

SEQ ID NO: 89 - protein, 12 aa, artificial sequence
  peptide recognition sequence retrieved from phage biopanning
FDNKPFLRVASE

SEQ ID NO: 90 - protein, 12 aa, artificial sequence
  peptide recognition sequence retrieved from phage biopanning
HQSHTQQNKRHL

SEQ ID NO: 91 - protein, 12 aa, artificial sequence
  peptide recognition sequence retrieved from phage biopanning
TSTTQGALAYLF

SEQ ID NO: 92 - protein, 12 aa, artificial sequence
  peptide recognition sequence retrieved from phage biopanning
KTPIHTSAWEFQ

SEQ ID NO: 93 - protein, 12 aa, artificial sequence
  peptide recognition sequence retrieved from phage biopanning
DLFHLKPVSNEK

SEQ ID NO: 94 - protein, 12 aa, artificial sequence
  peptide recognition sequence retrieved from phage biopanning
KPFWTSSPDVMT

SEQ ID NO: 95 - protein, 12 aa, artificial sequence
  peptide recognition sequence retrieved from phage biopanning
PWAATSKPPYSS